(19) United States
(12) Patent Application Publication
(10) Pub. No.: US 2026/0144483 A1
(43) Pub. Date: May 28, 2026

(54) CIRCADIAN RHYTHM DETECTION

(71) Applicant: Polar Electro Oy, KEMPELE (FI)

(72) Inventors: Riikka Ahola, KEMPELE (FI); Topi Korhonen, Kempele (FI); Matti Luomala, Kempele (FI); Kaisu Martinmäki, Kempele (FI); Lotta Rönnberg, Kempele (FI); Eve Vallström, Kempele (FI)

(73) Assignee: Polar Electro Oy, KEMPELE (FI)

(21) Appl. No.: 19/123,119

(22) PCT Filed: Nov. 2, 2023
(86) PCT No.: PCT/FI2023/050609

§ 371 Date: Apr. 22, 2025

(30) Foreign Application Priority Data
Nov. 4, 2022 (GB) 2216417.2

(51) Int. Cl. A61B 5/00; A61B 5/01

(52) U.S. Cl. CPC A61B 5/4857; A61B 5/01; A61B 5/4809; A61B 5/681; A61B 5/6844; A61B 5/742

(57) ABSTRACT

A method for detecting a circadian rhythm of a user, which includes acquiring measurement data indicating the user's sleep time and determining a user's sleep-wake rhythm of the user based on the measurement data; determining a first phase value based on the sleep-wake rhythm; acquiring skin temperature measurement data for at least one 24-hour measurement period, fitting the skin temperature measurement data to a cosine waveform, and performing evaluation of a quality of the fitting and, if the quality is above a threshold, determining a second phase value on the basis of the fitting; if the quality of the fitting is above the threshold, determining a circadian phase of the user on the basis of a combination of the first phase value and the second phase value; if the quality of the fitting is below the threshold, determining a circadian phase of the user based on the first phase value without the second phase value; and outputting the computed circadian phase or a parameter derivative of the computed circadian phase via an interface to the user.

### CROSS-REFERENCE TO RELATED APPLICATIONS

This application is a national phase application of International Application No. PCT/FI2023/050609, filed Nov. 2, 2023, which claims benefit and priority to Great Britain Application No. 2216417.2, filed Nov. 4, 2022, which are incorporated by reference herein in their entireties.

### BACKGROUND

This invention relates to detecting a user's circadian rhythm, i.e. user's sleep-wake rhythm.

### SUMMARY

A circadian rhythm (equivalently circadian process, circadian cycle) may be understood as a human internal physiological process regulating a natural sleep-wake rhythm of a person. The circadian rhythm repeats approximately every 24 hours. The circadian cycle comprises a circadian phase that can be understood as reflecting timing of different states of the circadian rhythm during a (calendar) day. Different persons have different natural circadian phases, and many everyday factors influence the person's capability and willingness to follow the natural circadian phase. With respect to the natural circadian phase, one person may be “an early bird” while another is “a night owl”. The night owl may have a job that forces the person to wake up earlier than the natural waking hour, while the early bird may have social activities that prolong the bedtime past a natural bedtime. As a consequence of the natural circadian phases and the fact that the user influences changes to the natural circadian phase, it is important to be capable of measuring the circadian phase. Automated measurement and detection of the circadian phase may be used as a basis for smart guidance features that benefit a user's health.

According to an aspect, there is a method for detecting a circadian rhythm of a user, the method comprising acquiring measurement data indicating the user's sleep time and determining a user's sleep-wake rhythm of the user on the basis of the measurement data; determining a first phase value based on the sleep-wake rhythm; acquiring skin temperature measurement data for at least one 24-hour measurement period; fitting the skin temperature measurement data to a cosine waveform, performing evaluation of a quality of the fitting and, if the quality is above a threshold, determining a second phase value on the basis of the fitting; if the quality of the fitting is above the threshold, determining a circadian phase of the user on the basis of a combination of the first phase value and the second phase value; if the quality of the fitting is below the threshold, determining the user's circadian phase of the user on the basis of the first phase value without the second phase value; and outputting the computed circadian phase or a parameter derivative of the computed circadian phase via an interface to be presented to the user.

According to an aspect, there is an apparatus for detecting a circadian rhythm of a user, the apparatus comprising acquiring measurement data indicating the user's sleep time and determining a user's sleep-wake rhythm of the user on the basis of the measurement data; determining a first phase value based on the sleep-wake rhythm; acquiring skin temperature measurement data for at least one 24-hour measurement period; fitting the skin temperature measurement data to a cosine waveform, and performing evaluation of a quality of the fitting and, if the quality is above a threshold, determining a second phase value on the basis of the fitting; if the quality of the fitting is above the threshold, determining a circadian phase of the user on the basis of a combination of the first phase value and the second phase value; if the quality of the fitting is below the threshold, determining the user's a circadian phase of the user on the basis of the first phase value without the second phase value; and outputting the computed circadian phase or a parameter derivative of the computed circadian phase via an interface to be presented to the user.

According to an aspect, there is a computer program product embodied on a distribution medium readable by a computer and comprising instructions, which, when loaded into an apparatus, execute detecting a circadian rhythm of a user, the computer program product comprising acquiring measurement data indicating the user's sleep time and determining a user's sleep-wake rhythm of the user on the basis of the measurement data; determining a first phase value based on the sleep-wake rhythm; acquiring skin temperature measurement data for at least one 24-hour measurement period; fitting the skin temperature measurement data to a cosine waveform, and performing evaluation of a quality of the fitting and, if the quality is above a threshold, determining a second phase value on the basis of the fitting; if the quality of the fitting is above the threshold, determining circadian phase of the user on the basis of a combination of the first phase value and the second phase value; if the quality of the fitting is below the threshold, determining the user's a circadian phase of the user on the basis of the first phase value without the second phase value; and outputting the computed circadian phase or a parameter derivative of the computed circadian phase via an interface to be presented to the user.

### BRIEF DESCRIPTION OF THE DRAWINGS

In the following the invention will be described in greater detail by means of preferred embodiments with reference to the attached drawings, in which

FIG. 1 illustrates an example system to which the embodiments of the invention may be applied;

FIG. 2 illustrates an embodiment of the method;

FIG. 3 illustrates an embodiment of the method wherein such skin temperature measurements data for which a corresponding skin contact measurement data indicates no contact to skin is excluded from fitting to the cosine waveform;

FIG. 4 illustrates an embodiment of the method wherein the amount of skin temperature measurement is checked against a first threshold;

FIG. 5 illustrates varying the phase of the cosine waveform;

FIG. 6 illustrates another embodiment of the method, wherein the circadian phase is chosen between first phase value and a combination of the first phase value and the second phase value;

FIG. 7 illustrates an embodiment of determining the circadian phase via sleep-wake rhythm; and

FIG. 8 illustrates an embodiment of an apparatus for detecting circadian rhythm;

### DETAILED DESCRIPTION

The following embodiments are exemplifying. Although the specification may refer to “an”, “one”, or “some” embodiment(s) in several locations of the text, this does not necessarily mean that each reference is made to the same embodiment(s), or that a particular feature only applies to a single embodiment. Single features of different embodiments may also be combined to provide other embodiments.

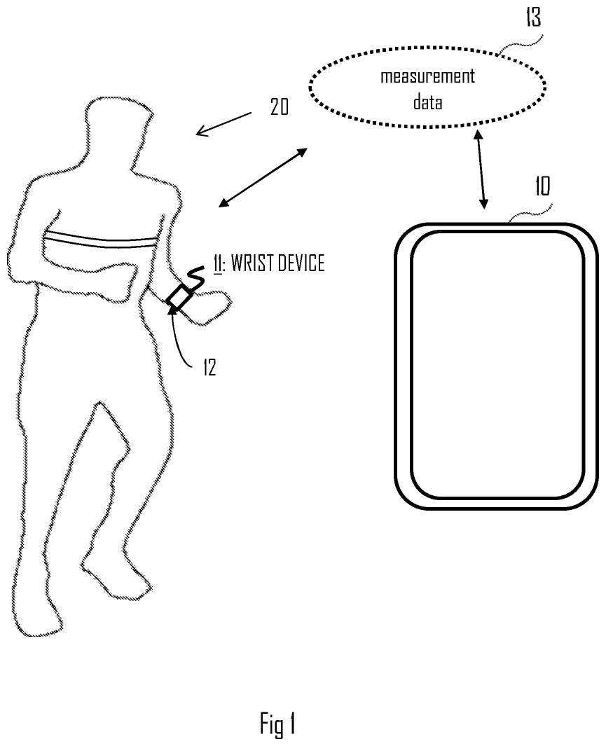

FIG. 1 illustrates a measurement system comprising a sensor device 12 that may be used in the context of some embodiments of the present invention. The user 20 may wear a wearable device, such as a wrist device 11. The wrist device 11 may be, for example, a smart watch, a smart device, sports watch, and/or an activity tracking apparatus (e.g. bracelet, arm band, wrist band, mobile phone, glasses). In an embodiment, the wrist device 11 is an activity tracking apparatus or a wearable activity tracking apparatus. This may mean that said apparatus may be worn in the wrist or in other parts of the user 20, such as but not limited to forearm, bicep area, neck, forehead, and/or leg. Data retrieved from wrist device 11 may also be used determine sleep start time and sleep end time, and skin temperature of the user 20. The embodiments described herein use wrist skin temperature and sleep start and wake-up times to determine circadian rhythm characteristics. Body core temperature represents the circadian rhythm in great detail, as known in the literature, and it peaks at daytime and is lowest at night. Therefore, it is possible to measure the circadian rhythm by measuring the body core temperature. The wrist skin temperature shows opposite patterns, being lowest at daytime and highest at night. However, they both follow the same rhythm and, therefore, it is possible to measure the circadian rhythm also from the skin temperature. A skin temperature sensor may be implemented in the wrist device 11 as one of sensors 12 of the wrist device 11.

In the example illustrated in FIG. 1, the measurement system comprises at least a processing circuitry configured to analyse measurement data 13 measured from the user 20, for example by carrying out methods described in more detail below. The processing circuitry may be realised in the wrist device 11 worn by the user, or the processing circuitry may be realised in a user device 10 such as a smart phone or a tablet computer, or the processing circuitry may be realised in a server computer such as a cloud server. The measurement data 13 may be provided by at least one sensor device 12 which may be comprised in the wrist device 11, or the sensor device 12 may be external to the wrist device 11 but provided with data transfer capability with the wrist device 11. The wrist device 11, the user device 102, the server computer, and/or the sensor device 12 may be connectable over one or more networks, over a short-range wireless connection such as Bluetooth, or over a Universal Serial Bus (USB) connection.

The sensors of the sensor device 12 may employ one or more measurement technologies for measuring the activity and skin temperature of user 20. The activity measurements may be based on using a heart activity sensor At least one sensor device 12 may be configured to measure the skin temperature, and also the skin contact using optical or bioimpedance method so that those temperature samples, which are measured when sensor is not worn, can be detected and optionally excluded from the estimation of the circadian rhythm. The sensor device 12 may measure one or more of the following features from the user: motion, electrocardiogram (ECG), photoplethysmogram (PPG), bioimpedance, galvanic skin response, body temperature. For measuring the motion, the sensor device 12 may comprise an inertial sensor such as an accelerometer and/or gyroscope, or a magnetometer, or any a sensor fusion that is any combination of these motion sensors, and the sensor device 12 may output motion measurement data. The sensor device 12 measuring ECG or PPG may output heart activity measurement data 13. The sensor device 12 may comprise one or more electrodes attachable to the user's 20 skin to measure an electric property from the skin which, through appropriate signal processing techniques, may be processed into an ECG signal, bioimpedance signal, or galvanic skin response signal. In some techniques, the heart activity measurement data 13 may represent appearance of R waves of electric heart impulses. In PPG measurements a light emitted by a light emitter diode or a similar light source and reflected back from the user's 20 skin is be sensed by using a photo diode or a similar light sensing component. The sensed light is then converted into an electric measurement signal in the light sensing component and signal processing is used to detect desired signal components from the electric measurement signal. In the PPG measurements, P waves may be detected which enables computation of a PP interval and a heart rate, for example.

The user device 10 refers to a computing device (equipment, apparatus) and it may also be referred to as a user terminal, a user apparatus, mobile device, or a mobile terminal. Portable computing devices (apparatuses) include wireless mobile communication devices operating with or without a subscriber identification module (SIM) in hardware or in software, including, but not limited to, the following types of devices: mobile phone, smartphone, personal digital assistant 30 (PDA), handset, laptop and/or touch screen computer, tablet (tablet computer), multimedia device, wearable computer, such as smart watch, and other types of wearable devices, such as clothing and accessories incorporating computer and advanced electronic technologies. The user device 102 may comprise one or more user interfaces. The one or more user interfaces may be any kind of a user interface, for example a screen, a keypad, a loudspeaker, a microphone, a touch user interface, an integrated display device, and/or external display device.

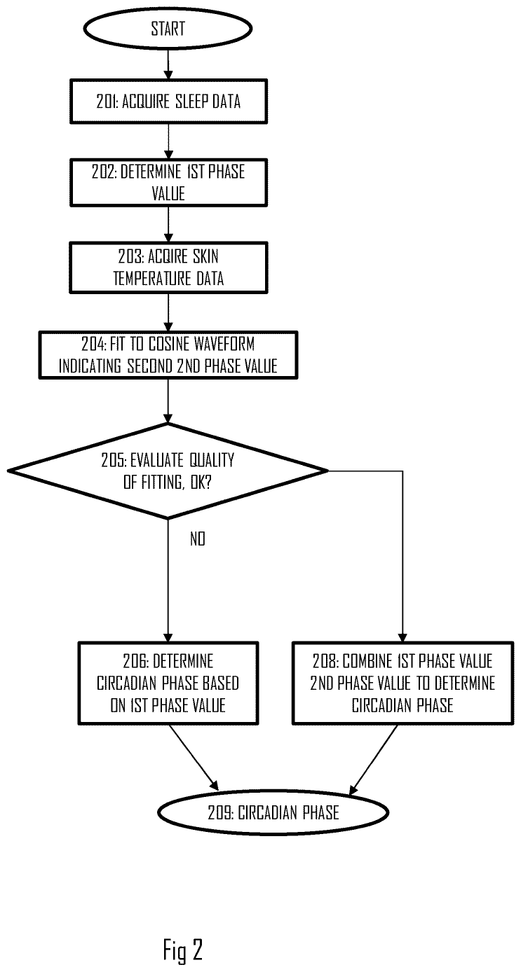

FIG. 2 is illustrating an embodiment, where there is a method for detecting a circadian rhythm of a user 20, wherein the method comprises acquiring 201 measurement data indicating the sleep time and determining a sleep-wake rhythm of the user 20 on the basis of the measurement data, and further determining 202 a first phase value based on the sleep-wake rhythm, and further acquiring 203 skin temperature measurement data for at least one 24-hour measurement period, and further fitting 204 the skin temperature measurement data to a cosine waveform, and performing evaluation 205 of a quality of the fitting and, if the quality is above a threshold, determining 207 a second phase value on the basis of the fitting, and if the quality of the fitting is above the threshold, determining 208 the user's 20 circadian phase as a combination of the first phase value and the second phase value, and if the quality of the fitting is below the threshold, determining 206 the user's 20 circadian phase based on the first phase value and outputting 209 the computed circadian phase or a parameter derivative of the computed circadian phase via an interface to be presented to the user 20. The second phase value can also be determined before evaluation 205 of the fitting 204 right after the fitting 204.

The measurement data acquired in block 201 may be acquired by using at least one of a heart activity sensor and a motion sensor of the sensor devices 12. The detection of the sleep start time and the sleep end time may be carried out according to the state of the art. The detection of the sleep start time and the sleep end time by using the heart activity sensor and/or the motion sensor is as such generally well known and there exist several commercially available products for performing this, e.g. Polar Vantage series by Polar Electro.

The embodiment of FIG. 2 provides the advantage of validating the second phase value that is based on the skin temperature measurements. Measuring the skin temperature requires skin contact between the sensor device 12 and the user's 20 skin. The user may wish to wear the sensor device 12 with a comfortable fit that does not press the sensor device 12 against the skin. The downside is that the skin contact may not be optimal. Furthermore, the skin contact may change during the daily activities of the user and during the night as well. Furthermore, while the fitting the skin temperature measurement data with the cosine function (also called cosinor analysis or cosinor fitting in the literature) is generally used for the circadian phase detection, the fitting may fail for several reasons. As a consequence, while the second estimate for the circadian phase improves the accuracy of the circadian phase estimation, it is advantageous to validate it before combining it with the first estimate.

In the procedure of FIG. 2, the first phase value is used for estimating the circadian phase, either alone or in combination with the second phase value. If the quality of the second phase value is determined to be high enough, e.g. the fitting is considered successful, the second phase value may be computed or the already-computed phase value may be considered eligible for the combining.

The measurement period should be at least one sleep-wake cycle i.e. 24 hours, but also a longer measurement period of several sleep-wake cycles provides better quality data. A measurement period of a week i.e. seven several sleep-wake cycles provides already very reliable data.

In an embodiment, the cosinor fitting in block 204 is performed by using least squares (LS) fitting known as such in the art. The LS fitting finds a cosine waveform (phase) that provides the lowest squared error with the measurement data. The phase of the fitted cosine waveform then represents the second phase value.

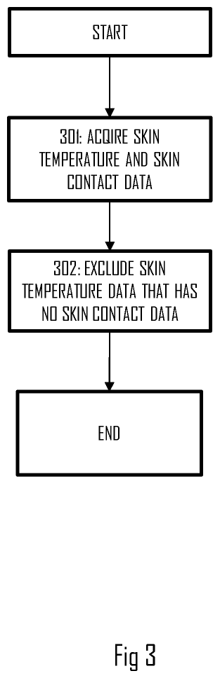

In an embodiment, the procedure of FIG. 2 comprises excluding from fitting to the cosine waveform such skin temperature measurement data for which a corresponding skin contact measurement data indicates no contact to skin. FIG. 3 is illustrating this embodiment, wherein skin contact measurement data is acquired 301 together with skin temperature measurement data for the at least one 24-hour measurement period, and such skin temperature measurements data—for which the skin contact measurement data indicating no contact to skin—is excluded 302 from the fitting to the cosine waveform in 204. As described above, a part of the skin temperature measurement data may be missing because the user 20 is not wearing the device at all for a while during the measurement period, or the device has no actual contact to user's 20 skin. The temperature measurement data associated with the lack of skin contact may indicate the ambient temperature that would degrade the performance of the fitting.

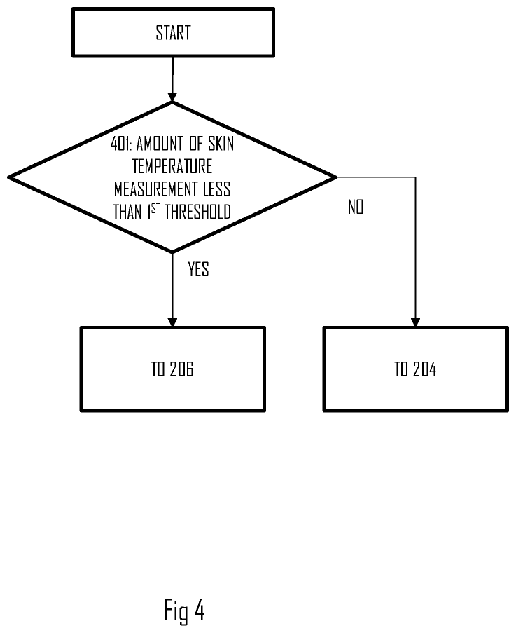

With respect to validating the skin temperature measurements and the second phase value, FIG. 4 is illustrating an embodiment of the method. Referring to FIG. 4, the procedure excluding 401 fitting of the skin temperature measurement data with the cosine waveform, if an amount of valid skin temperature measurement data associated with the skin contact measurement data indicating the skin contact is less than a first threshold during the at least one 24-hour skin temperature measurement period. In an embodiment, valid skin temperature measurement data covers at least 80% of the maximum skin temperature measurement data. This means that with at least 80% of the measurements within the 24-hour cycle the skin contact shall be present. For example, if the sampling rate of temperature measurements is one measurement per five minutes (288 measurement samples per 24-hour cycle), there should be at least 231 validated measurement samples per 24-hour cycle. Having less than 80% of the skin temperature measurement data may be considered to reduce the reliability of circadian rhythm detection using the skin temperature measurement. In other embodiments, lower percentages may be allowed, depending how much error the implementation tolerates and how accurate the first phase value is determined to be. According to Nyquist criterion, two samples per 24-hour cycle is a minimum but, in reality, greater numbers of measurement samples per 24-hour cycle are preferred to improve the accuracy of the second estimate. Instead of the percentage value, an absolute value for the minimum number of validated measurements with the detected skin contact may be defined. Such a value may be, for example, 50 or 100 or 150 or 200 per 24-hour cycle.

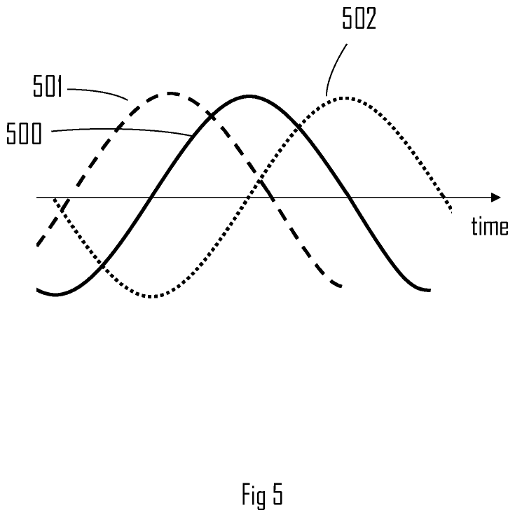

In another embodiment, the skin temperature measurements are validated by evaluating the performance of the cosinor fitting of block 204. This may be carried out by arranging a phase shift between the measurement data and the cosine waveform after the fitting and evaluating a change in an error metric indicating error between the measurement data and the cosine waveform. In other words, the phase shifting is used to determine whether or not there is a better fit than that selected in 204. FIG. 5 is illustrating waveforms used in the quality evaluation of the second phase value after the fitting of the skin temperature measurement data with the cosine waveform. In this embodiment evaluating the quality of the fitting comprises the following steps
determining a first correspondence metric for the skin temperature measurement data with respect to the cosine waveform 500 after the fitting. The first correspondence metric indicates the error between the skin temperature measurement data with respect to the cosine waveform 500 after the fitting.
arranging a phase shift forward 502 and/or backward 501 between the cosine waveform 500 and the skin temperature measurement data fitted with the cosine waveform 500 and determining at least a second correspondence metric for the skin temperature measurement data with respect to the cosine waveform after the phase shift(s) 501, 502. In case multiple phase shifts are performed, multiple correspondence metrics are computed.
determining that the fit quality is above the threshold, if the second correspondence metric (and further correspondence metric(s)) indicates a greater error than the first correspondence metric, and determining that the fit quality is below the threshold, if the second correspondence metric (or at least one of the further correspondence metric(s)) indicates a smaller error than the first correspondence metric.

In other words, if the phase shift results in a fitting associated with the lower fitting error, the fitting performed in 204 has not been optimal and the circadian phase may be determined in block 206 by using only the sleek-wake rhythm. The calculation of the correspondence metrics may be done by calculating a root-mean-square-error (RMSE) of the fit i.e. the RMSE between the skin temperature measurement data and the fitted cosine waveform. Similarly, the RMSE may be calculated between the skin temperature measurement data and the shifted cosine waveforms in the case of the second correspondence metric. The RMSE should increase when the phase of the cosine waveform is varied, thus indicating that the fitting has been optimal and the phase of the fitted cosine waveform can be validated as the circadian phase estimate. The absolute value of RMSE can vary a lot between different users, but the essential thing is that the RMSE should increase when the fitted phase value is varied, forward or backward. The quality evaluation may also be carried out using a different calculation of the corresponding metrics, such as a mean-absolute-error (MAE). The correspondence metric is preferably different from the metric used for the fitting in block 204.

The result from second phase value can be ignored or its computation may be omitted, if there are too few temperature samples with verified skin contact available or if the quality evaluation analysis (e.g. RMSE based calculation) of fitted second phase value reveals that the fit was not very accurate.

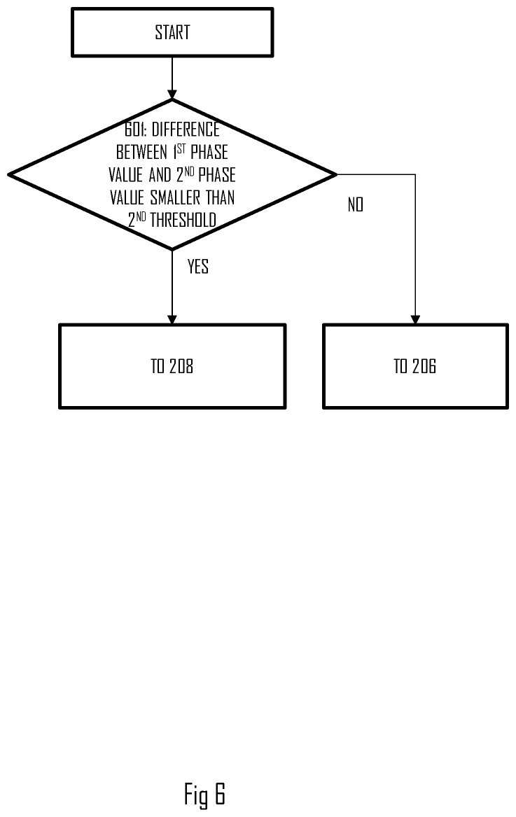

FIG. 6 is illustrating yet another embodiment for validating the second phase value, wherein the method further comprises checking 601 that if a difference between the first phase value and the second phase value is smaller than a second threshold, the circadian phase is determined 602 as a combination of the first phase value and the second phase value. The combination may be average or a weighted average of the first phase value and the second phase value. In another embodiment, the first phase value is shifted on the basis of the difference between the first phase value and the second phase value. A maximum limit for the shift may have been defined, e.g. four hours. The shifting may distinguish from the averaging operation and be linear or non-linear with respect to the difference between the first phase value and the second phase value. If the difference between the first phase value and the second phase value is greater than a second threshold, the circadian phase is determined as the first phase value. The second threshold may be defined in terms of a time shift between the first and second phase value. Example values of the second threshold may range from one hour to even ten hours e.g. 1, 2, 3, 4, 5, 6, 7, 8, 9 or 10 hours. Combining this with the embodiment where the first phase value is shifted on the basis of the difference, if the second threshold is 10 hours and the maximum shift is 4 hours, the first phase value may be shifted by 4 hours towards the second phase value when the difference is 10 hours. If the first and second phase value are equal, no shifting of the phase value is performed. The shifts when the difference is between zero and 10 hours may then be mapped in a linear or non-linear manner.

As described above, the first phase value is computed from the sleep-wake rhythm that may be based on heart activity measurement data and/or motion measurement data, while the second phase value is computed on the basis of the skin temperature measurement data fitted with the cosine waveform. While both phase values indicate the circadian phase, they are inherently in different domains because of the different measurement data and different physiological characteristics measured. Therefore, an embodiment comprises shifting one of the first phase value and the second phase value by a constant value to make the first phase value and the second phase value comparable.

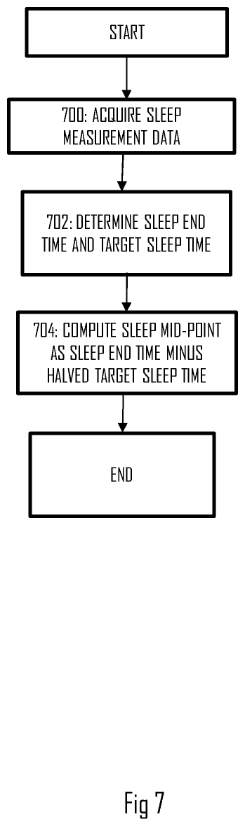

Conventional methods for estimating the circadian phase from the sleep-wake rhythm are based on determining a sleep mid-point as a mid-point between a sleep start time and sleep end time detected from the measurement data. This provides an accurate circadian phase estimate when the user's 20 sleep rhythm is regular. However, if the user reduces the sleep time by going to sleep later and waking up at the same time, the sleep mid-point gets delayed and the circadian phase is also shifted (delayed). The problem is that in such a case the user's 20 circadian phase does not actually change, but the user gains sleep debt. To overcome the problem of the user 20 changing his/her sleep times, e.g. reducing the amount of sleep but waking at the same time, an embodiment omits the sleep start time from the estimation of the first phase value. Instead, the first phase value is computed on the basis of a target sleep time (required amount of sleep which may be estimated e.g. with a sleep assistant algorithm, general sleep amount guidelines, or received as a user input), together with the sleep end time to estimate what the sleep mid-point would have been if the user 20 would have slept enough before waking up. This method provides a more accurate circadian phase estimate in a situation where the user is generating sleep debt with his/her sleeping habits. This may be realized by acquiring a sleep end time from the measurement data indicating the user's 20 sleep time and further acquiring a target sleep goal time of the user 20, determining a sleep mid-point of the user 20 on the basis of the sleep end time and the target sleep time, and computing the sleep-wake rhythm on the basis of the sleep mid-point. FIG. 7 illustrates a flow diagram of an embodiment for computing the sleep mid-point for the purpose of estimating the circadian rhythm. Referring to FIG. 7, the sleep measurement data is acquired in 700. The sleep measurement data may comprise the heart activity measurement data and/or motion measurement data that enable detection of at least the sleep end time. In 702, the sleep end time and the target sleep time are determined. The target sleep time may have been stored in the memory beforehand, and the sleep end time may be determined from the sleep measurement data acquired in 700. The sleep mid-point may be determined as a mid-point between the sleep end time and the sleep end time minus the halved target sleep time according to the following Equation in 704

$$sleepMidPointEstimate = sleepEndTime - TargetSleepTime / 2 \qquad ( 1 )$$

The purpose of computing the circadian phase may be to provide the user with information related to the circadian phase. Such information may include, for example, recommending a bedtime to the user. As known in the art, the circadian rhythm represents an internal clock and thus carries information on a natural bedtime for the user. If the user follows his/her natural rhythm, health benefits may be achieved. Another example of a parameter derivative of the circadian phase is the user's 20 chronotype. The computation of the circadian phase enables classifying a sleep chronotype of the user 20 on the basis of the circadian phase and outputting, as the parameter, the sleep chronotype via the interface. The chronotype classification may be based on observing the user's 20 circadian phase for a prolonged period of time, e.g. at least a week. The circadian phase of the night owl may be delayed with respect to the circadian phase of the early bird, for example. Yet another example of a parameter derivative of the circadian phase is an alarm of reduced alertness output to the user. For example, if the circadian phase estimation indicates that the user's 20 circadian rhythm is changing as a result of travelling across time zones or as a result of shift work, the user may be alerted that it has consequences in the form of reduced alertness. The parameters listed above may be displayed to the user via a user interface of the wrist device 11 or the user device 10, for example.

The above-described embodiments for determining the circadian phase may be used to directly infer the circadian phase or to infer a setpoint for the circadian phase. If the user lives a regular daily rhythm including a regular sleep cycle by entering sleep and waking up substantially at the same time each day, the above-described embodiments may directly indicate the user's 20 circadian phase. However, if the user changes his/her sleep-wake rhythm as a result of travelling over time zones, shift work etc., it takes time for the circadian phase to adapt to the new sleep-wake rhythm. In such a case, the above-described embodiments may define a setpoint for the circadian phase towards which the circadian phase adopts at a certain daily adaptation rate. In such a case, the circadian phase for today may be determined on the basis of the setpoint, the adaptation rate, and the circadian phase yesterday. As a consequence, the above-described embodiments may be used for determining the circadian phase in a case where the circadian phase is adapting. The adaptation rate may be a constant or it may be a function of whether the circadian phase is shifting clockwise (delaying) or counter-clockwise (advancing). The clockwise adaptation rate (e.g. one hour per day) may be greater than the counter-clockwise adaptation rate (e.g. 0.67 hours per day).

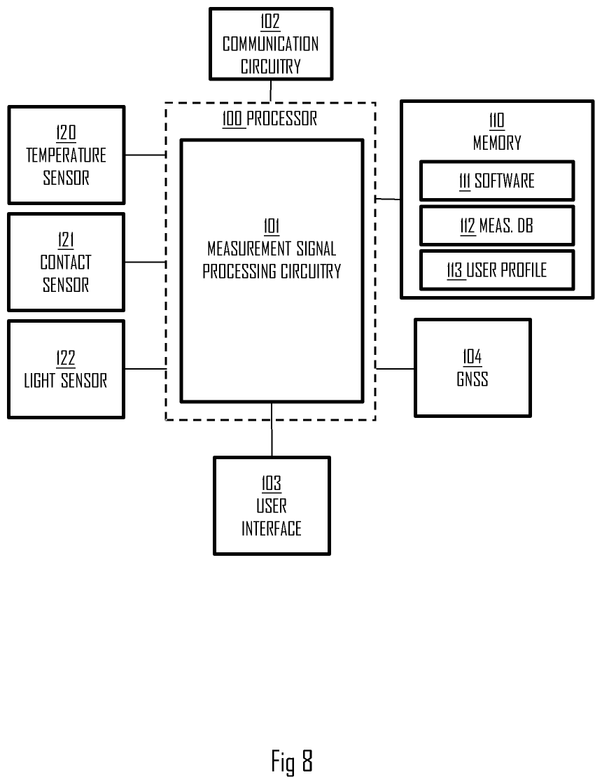

FIG. 8 illustrates an embodiment of an apparatus configured to carry out at least some of the above-described functions in detecting a user's 20 circadian rhythm. The apparatus may comprise an electronic device comprising at least one processor 100 and at least one memory 110. The processor 100 may form or be a part of a processing circuitry. The apparatus may further comprise a user interface 103 comprising a display screen or another display unit, an input device such as one or more buttons and/or a touch-sensitive surface, and an audio output device such as a loudspeaker.

The processor 100 may comprise a measurement signal processing circuitry 101 configured to detect the user's 20 circadian rhythm by carrying out the procedure of FIG. 2 or any one of its embodiments described herein.

The apparatus may comprise a communication circuitry 102 connected to the processor 100. The processor 100 may use the communication circuitry 102 to transmit and receive frames according to the supported wireless communication protocol. In some embodiments, the processor 100 may use the communication circuitry 102 to transmit the data on the user's 20 circadian rhythm, sleep data and/or other parameters to another apparatus, e.g. to a cloud server storing the user's 20 user account.

In an embodiment, the apparatus comprises at least one skin temperature sensor 120. Additionally, the apparatus may comprise at least one skin contact sensor 121. The skin contact sensor 121 may use optical or bioimpedance method. In an embodiment, the apparatus comprises a light sensor 122 to measure the light exposure and further determine the sun-light rhythm of the user's 20 location.

In the embodiments, the devices may comprise a global positioning system or circuitry 104 such as the Global Positioning System or another satellite navigation system (generally a global navigation satellite system, GNSS) to provide location information to be used in determining the user's 20 location. The apparatus may also receive the location information from the internet using the communication circuitry 102.

The memory 110 may store a computer program product 111 circadian rhythm detection algorithm the processor executes upon reading the computer program. The memory may further store a user profile 113 of the user 20 storing personal characteristics of the user 20. The memory may further store a measurement database 112 comprising the measured history of sleep-wake rhythm, circadian rhythm, and user's 20 preferences or plans related to events such as travelling over time zones, daylight saving time transitions or shift work schedules.

As used in this application, the term ‘circuitry’ refers to all of the following: (a) hardware-only circuit implementations, such as implementations in only analog and/or digital circuitry, and (b) combinations of circuits and software (and/or firmware), such as (as applicable): (i) a combination of processor(s) or (ii) portions of processor(s)/software including digital signal processor(s), software, and memory(ies) that work together to cause an apparatus to perform various functions, and (c) circuits, such as a microprocessor(s) or a portion of a microprocessor(s), that require software or firmware for operation, even if the software or firmware is not physically present. This definition of ‘circuitry’ applies to all uses of this term in this application. As a further example, as used in this application, the term ‘circuitry’ would also cover an implementation of merely a processor (or multiple processors) or a portion of a processor and its (or their) accompanying software and/or firmware. The term ‘circuitry’ would also cover, for example and if applicable to the particular element, a baseband integrated circuit or applications processor integrated circuit for a mobile phone or a similar integrated circuit in a server, a cellular network device, or another network device.

In an embodiment, at least some of the processes described in connection with FIGS. 2-7 may be carried out by an apparatus comprising corresponding means for carrying out at least some of the described processes. Some example means for carrying out the processes may include at least one of the following: detector, processor (including dual-core and multiple-core processors), digital signal processor, controller, receiver, transmitter, encoder, decoder, memory, RAM, ROM, software, firmware, display, user interface, display circuitry, user interface circuitry, user interface software, display software, circuit, and circuitry. In an embodiment, the at least one processor 100, the memory 110, and the computer program code 118 form processing means or comprises one or more computer program code portions for carrying out one or more operations according to any one of the embodiments of FIGS. 2-4 and 6 or operations thereof.

The techniques and methods described herein may be implemented by various means. For example, these techniques may be implemented in hardware (one or more devices), firmware (one or more devices), software (one or more modules), or combinations thereof. For a hardware implementation, the apparatus(es) of embodiments may be implemented within one or more application-specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described herein, or a combination thereof. For firmware or software, the implementation can be carried out through modules of at least one chipset (e.g. procedures, functions, and so on) that perform the functions described herein. The software codes may be stored in a memory unit and executed by processors. The memory unit may be implemented within the processor or externally to the processor. In the latter case, it can be communicatively coupled to the processor via various means, as is known in the art. Additionally, the components of the systems described herein may be rearranged and/or complemented by additional components in order to facilitate the achievements of the various aspects, etc., described with regard thereto, and they are not limited to the precise configurations set forth in the given figures, as will be appreciated by one skilled in the art.

Embodiments as described may also be carried out in the form of a computer process defined by a computer program or portions thereof. Embodiments of the methods described in connection with FIGS. 2-4 and 6 may be carried out by executing at least one portion of a computer program comprising corresponding instructions. The computer program may be in source code form, object code form, or in some intermediate form, and it may be stored in some sort of carrier, which may be any entity or device capable of carrying the program. For example, the computer program may be stored on a computer program distribution medium readable by a computer or a processor. The computer program medium may be, for example but not limited to, a record medium, computer memory, read-only memory, electrical carrier signal, telecommunications signal, and software distribution package, for example. The computer program medium may be a non-transitory medium. Coding of software for carrying out the embodiments as shown and described is well within the scope of a person of ordinary skill in the art.

It will be obvious to a person skilled in the art that, as the technology advances, the inventive concept can be implemented in various ways. The invention and its embodiments are not limited to the examples described above but may vary within the scope of the claims.

What is claimed is:

1. A method for detecting a circadian rhythm of a user, the method comprising:
acquiring measurement data indicating the user's sleep time and determining a sleep-wake rhythm of the user on the basis of the measurement data;
determining a first phase value based on the sleep-wake rhythm;
acquiring skin temperature measurement data for at least one 24-hour measurement period;
fitting the skin temperature measurement data to a cosine waveform, performing evaluation of a quality of the fitting and, if the quality is above a threshold, determining a second phase value on the basis of the fitting;
if the quality of the fitting is above the threshold, determining a circadian phase of the user on the basis of a combination of the first phase value and the second phase value;
if the quality of the fitting is below the threshold, determining the user's circadian phase of the user on the basis of the first phase value without the second phase value; and
outputting the computed circadian phase or a parameter derivative of the computed circadian phase via an interface to be presented to the user.

2. The method according to claim 1, further comprising:
acquiring skin contact measurement data together with skin temperature measurement data for the at least one 24-hour measurement period; and
excluding from fitting to the cosine waveform such skin temperature measurement data for which a corresponding skin contact measurement data indicates no contact to skin.

3. The method according to claim 2, further comprising excluding the fitting of the skin temperature measurement data if an amount of skin temperature measurement data associated with skin contact measurement data indicating a skin contact is less than a first threshold during the at least one 24-hour skin temperature measurement period.

4. The method according to claim 3, wherein the first threshold is 80% of the skin temperature measurement data.

5. The method according to claim 1, wherein said performing the evaluation of the quality of the fitting further comprises:
determining a first correspondence metric for the skin temperature measurement data with respect to the cosine waveform after the fitting;
arranging a phase shift between the cosine waveform and the skin temperature measurement data fitted to the cosine waveform and determining a second correspondence metric for the skin temperature measurement data with respect to the cosine waveform after the phase shift; and
determining that the fit quality is above the threshold, if the second correspondence metric indicates greater error than the first correspondence metric, and determining that the fit quality is below the threshold, if the second correspondence metric smaller error than the first correspondence metric.

6. The method according to claim 5, wherein the first correspondence metric represents a root mean square error or a mean absolute error between the skin temperature measurement data and the cosine waveform after the fitting; and the second correspondence metric represents a root mean square error or a mean absolute error between the skin temperature measurement data and the cosine waveform after the phase shift.

7. The method according to claim 1, further comprising determining the user's circadian phase as a combination of the first phase value and the second phase value, if a difference between the first phase value and the second phase value is smaller than a second threshold and determining the user's circadian phase as the first phase value, if the difference between the first phase value and the second phase value is greater than the second threshold.

8. The method according to claim 7, wherein said combination is performed by shifting the first phase value towards the second phase value in linear or non-linear proportion to the difference between the first phase value and the second phase value.

9. The method according to claim 1, further comprising shifting one of the first phase value and the second phase value by a constant value to make the first phase value and the second phase value comparable.

10. The method according to claim 1, further comprising acquiring a sleep end time from the measurement data indicating the user's sleep time and further acquiring a target sleep time of the user, determining a sleep mid-point of the user on the basis of the sleep end time and the target sleep time, and computing the sleep-wake rhythm on the basis of the sleep mid-point.

11. The method of claim 9, wherein the sleep mid-point is determined as a mid-point between the sleep end time and the sleep end time minus the target sleep time.

12. The method of claim 1, further comprising classifying a sleep chronotype of the user on the basis of the circadian phase and outputting, as the parameter, the sleep chronotype via the interface.

13. An apparatus for detecting a circadian rhythm of a user, comprising:
acquiring measurement data indicating the user's sleep time and determining a user's sleep-wake rhythm of the user on the basis of the measurement data;
determining a first phase value based on the sleep-wake rhythm;
acquiring skin temperature measurement data for at least one 24-hour measurement period;
fitting the skin temperature measurement data to a cosine waveform, and performing evaluation of a quality of the fitting and, if the quality is above a threshold, determining a second phase value on the basis of the fitting;
if the quality of the fitting is above the threshold, determining the user's a circadian phase of the user as a combination of the first phase value and the second phase value;
if the quality of the fitting is below the threshold, determining the user's a circadian phase of the user on the basis of the first phase value without the second phase value; and
outputting the computed circadian phase or a parameter derivative of the computed circadian phase via an interface to be presented to the user.

14. The apparatus according to claim 13, further comprising means to carry out operations comprising:
acquiring skin contact measurement data together with skin temperature measurement data for the at least one 24-hour measurement period; and
excluding from fitting to the cosine waveform such skin temperature measurement data for which a corresponding skin contact measurement data indicates no contact to skin.

15. A computer program product embodied on a distribution medium readable by a computer and comprising instructions that when executed by an apparatus, perform operations comprising:
detecting a circadian rhythm of a user, comprising:
acquiring measurement data indicating the user's sleep time and determining a user's sleep-wake rhythm of the user on the basis of the measurement data;
determining a first phase value based on the sleep-wake rhythm;
acquiring skin temperature measurement data for at least one 24-hour measurement period;
fitting the skin temperature measurement data to a cosine waveform, and performing evaluation of a quality of the fitting and, if the quality is above a threshold, determining a second phase value on the basis of the fitting;
if the quality of the fitting is above the threshold, determining the user's a circadian phase of the user as a combination of the first phase value and the second phase value;
if the quality of the fitting is below the threshold, determining the user's a circadian phase of the user on the basis of the first phase value without the second phase value; and
outputting the computed circadian phase or a parameter derivative of the computed circadian phase via an interface to be presented to the user.

16. A computer program product according to claim 15 embodied on a distribution medium readable by a computer and comprising instructions that, when executed by an apparatus, perform operations comprising:
acquiring skin contact measurement data together with skin temperature measurement data for the at least one 24-hour measurement period; and
excluding from fitting to the cosine waveform such skin temperature measurement data for which a corresponding skin contact measurement data indicates no contact to skin.
